(19) United States
(12) Patent Application Publication
(10) Pub. No.: US 2018/0085143 A1
(43) Pub. Date: Mar. 29, 2018

(54) APPARATUS FOR HAIR TRANSPLANTATION

(71) Applicants: James B. DeYarman, La Jolla, CA (US); David G. Matsuura, Del Mar, CA (US); Belinko K. Matsuura, Encinitas, CA (US)

(72) Inventors: James B. DeYarman, La Jolla, CA (US); David G. Matsuura, Del Mar, CA (US); Belinko K. Matsuura, Encinitas, CA (US)

(21) Appl. No.: 15/718,454

(22) Filed: Sep. 28, 2017

Related U.S. Application Data
(60) Provisional application No. 62/401,760, filed on Sep. 29, 2016.

(51) Int. Cl. A61B 17/34

(52) U.S. Cl. CPC A61B 17/3468; A61B 2017/00752; A61B 17/3496; A61B 17/3417

(57) ABSTRACT

Systems, methods, and apparatuses are disclosed for transplanting hair. In some example embodiments, an apparatus for transplanting hair may include a handle, a needle extending from the handle and including a beveled distal end for creating an implant site in skin. The implant site may be configured to receive a follicular unit having at least one hair follicle. The apparatus may further include a shuttle tube that extends from the handle along a first lumen of the needle. The shuttle tube may be configured to capture the follicular unit for implanting in the implant site. The apparatus may further include a stabilizer that extends from the handle along a second lumen of the shuttle tube. The stabilizer may stabilize the follicular unit in the implant site.

### REFERENCE TO PRIORITY DOCUMENT

The present application claims priority to co-pending U.S. Provisional Application Ser. No. 62/401,760 entitled “APPARATUS FOR HAIR TRANSPLANTATION” and filed Sep. 29, 2016. Priority of the aforementioned filing date is claimed and the provisional application is incorporated by reference in its entirety.

### FIELD

The subject matter described herein relates to an apparatus and method for transplanting hair.

### BACKGROUND

Hair transplantation is an expanding medical need and growing business. A variety of techniques may be used to transplant hair. Most involve taking hair-bearing skin from one part of the scalp and grafting the hair-bearing skin to a bald or thinning area of the scalp or area where injury has occurred. Hair loss may be due to any of a number of problems including male pattern baldness, scalp inflammation, or injury. Injuries may leave permanent areas of hair loss that can be concealed by hair transplantation. Improved surgical tools are needed to more efficiently, reliably, and cost-effectively provide hair transplantation to patients.

### SUMMARY

Systems, methods, and apparatuses are disclosed for transplanting hair. In some example embodiments, an apparatus for transplanting hair may include a handle, a needle extending from the handle including a beveled distal end for creating an implant site in skin. The implant site may be configured to receive a follicular unit having at least one hair follicle. The apparatus may further include a shuttle tube that extends from the handle along a first lumen of the needle. The shuttle tube may be configured to capture the follicular unit for implanting in the implant site. The apparatus may further include a stabilizer that extends from the handle along a second lumen of the shuttle tube. The stabilizer may stabilize the follicular unit in the implant site.

In some implementations, the above-noted aspects may further include additional features described herein including one or more of the following in any combination. The needle may include markings along a circumference of the needle to indicate a needle insertion depth in the skin. The beveled distal end may include a first beveled angle extending a first distance from the distal end and a second beveled angle extending a second distance from a proximal end of the first beveled angle. A distal end of the handle may include a dial to threadably control a length the needle extends from the handle. The shuttle tube may include a longitudinal opening extending a length from a distal end of the shuttle tube, and/or the longitudinal opening may be shaped to allow the follicular unit being grasped by a grasping device to be loaded within the second lumen of the shuttle tube through the longitudinal opening. The shuttle tube may include a transverse opening extending a partial circumference around the shuttle tube for allowing release of the grasping device from the follicular unit after loading the follicular unit in the second lumen of the shuttle tube. The apparatus may further include more than one needle to implant more than one follicular unit at approximately the same time. The needle may retract the skin to protect the implant site during the implanting of the follicular unit. The hair transplantation apparatus may be attached to a medical robot or a hair transplantation robot and/or controlled by the robot.

The above-noted aspects and features may be implemented in systems, apparatus, methods, and/or articles depending on the desired configuration. The details of one or more variations of the subject matter described herein are set forth in the accompanying drawings and the description below. Features and advantages of the subject matter described herein will be apparent from the description and drawings, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of this specification, show certain aspects of the subject matter disclosed herein and, together with the description, help explain some of the principles associated with the disclosed implementations. In the drawings,

FIG. 1 illustrates an embodiment of a hair transplantation device including a needle with a shuttle in a retracted position;

FIG. 2 illustrates the hair transplantation device of FIG. 1 showing the needle with the shuttle extended to a loading position;

FIG. 3 illustrates the hair transplantation device of FIG. 1 showing a detail view of a shuttle in a loading position;

FIG. 4 illustrates the hair transplantation device of FIG. 1 showing a follicular unit, shuttle, and needle;

FIG. 5 illustrates the hair transplantation device of FIG. 1 showing a follicular unit loaded into a shuttle and needle;

FIG. 6 illustrates the hair transplantation device of FIG. 1 showing a cross-section view of a follicular unit loaded into a needle;

FIG. 7 illustrates the hair transplantation device of FIG. 1 showing a shuttle retracted;

FIG. 8 illustrates the hair transplantation device of FIG. 1 showing a detail view of a needle with a shuttle retracted;

FIG. 9 illustrates the hair transplantation device of FIG. 1 showing a cross-section view of a needle with a shuttle retracted;

FIG. 10 illustrates the hair transplantation device of FIG. 1 showing the distal end of a needle inserted into skin;

FIG. 11 illustrates the hair transplantation device of FIG. 1 showing the distal end of a needle inserted into skin;

FIG. 12 illustrates the hair transplantation device of FIG. 1 showing a needle inserted into skin;

FIG. 13 illustrates the hair transplantation device of FIG. 1 showing a cross section view of a needle inserted into skin;

FIG. 14 illustrates the hair transplantation device of FIG. 1 showing a follicular unit advanced into skin with an outer sheath against the surface of the skin;

FIG. 15 illustrates the hair transplantation device of FIG. 1 showing a detail view of a follicular unit advanced into skin;

FIG. 16 illustrates the hair transplantation device of FIG. 1 showing a cross section view of a follicular unit advanced into skin with an outer sheath against the surface of the skin;

FIG. 17 illustrates the hair transplantation device of FIG. 1 showing a shuttle retracted into a needle, and the shuttle and needle retracted into a handle;

FIG. 18 illustrates the hair transplantation device of FIG. 1 showing a detail view of an implanted follicular unit with a needle retracted into a shuttle;

FIG. 19 illustrates the hair transplantation device of FIG. 1 showing a cross section view of an implanted follicular unit with a needle retracted into a shuttle;

FIG. 20 illustrates the hair transplantation device of FIG. 1 showing a retracted needle;

FIG. 21 illustrates an example of a follicular unit implanted into skin by the hair transplantation device of FIG. 1; and

FIGS. 22-24 illustrates another embodiment of the hair transplantation device having multiple needles for transplanting more than one follicular unit at the same or approximately the same time.

Like labels are used to refer to same or similar items in the drawings.

### DETAILED DESCRIPTION

A hair transplantation device is disclosed that prepares an implant site in skin followed by implanting a follicular unit into the prepared site. The hair transplantation device can include a beveled outer cannula at a distal end of the device that prepares an implant site. For example, the beveled outer cannula, or needle, may be inserted into the scalp or other skin of a patient to a predetermined depth. The inserted needle may create an opening in the skin that is protected by the needle. An inner second cannula, or shuttle, may be inside the needle. The shuttle may have a hair follicle, or follicular unit, inside the shuttle. The shuttle may also be beveled at the distal end with the follicular unit extending into the beveled end of the shuttle. The shuttle may be translated along the needle in a distal direction to deliver the follicular unit to the implant site. The shuttle may then be retracted. The needle and shuttle may be shaped to allow friction between the follicular unit and the tissue/skin at the implant site to hold the follicular unit in place while the shuttle is retracted. The follicular unit may extend into the beveled end of the shuttle to allow contact between the tissue/skin and the follicular unit to generate the friction. A stabilizer in the shuttle positioned proximally from the beveled end may stabilize the follicular unit in the implant site as the shuttle is retracted and the follicular unit is implanted. In some embodiments, the device can prepare one implant site and one follicular unit may be implanted per use of the device. In other embodiments, the device can prepare more than one site and more than one follicular unit may be implanted per use of the device. In some embodiments, the hair transplantation device may be attached to a medical robot or to a hair transplantation robot. The robot may control one or more aspects of the hair transplantation device. For example, a medical robot may position the hair transplantation device, may actuate implantation, may load follicular units, and so on.

The hair transplantation device may include a handle. A surgeon or operator of the device may grasp the handle with their hand. The handle may be cylindrical or formed into another shape with the shuttle, stabilizer, needle, and other elements of the device coupled to and extending from the handle. The handle may be made from plastic, metal, or any other material, or combination of materials. The handle has a proximal end that is grasped by the surgeon/operator and a distal end for hair transplantation. The handle may include one or more openings along the length of the handle and/or at the proximal end of the handle for operating the device. For example, openings along the length of the handle may allow operator controls such as a needle retractor and/or a shuttle retractor to exit the handle. At the proximal end of the device (or elsewhere on the device) a depth adjuster may allow for the operator to adjust the depth that the needle is inserted into the skin.

The hair transplantation device may include a needle. The needle may be cylindrical or formed into any other shape. The needle may have an outer diameter between 0.5 mm and 2.0 mm, with a lumen diameter between 0.5 mm and 1.5 mm. A follicular unit may have a diameter of approximately 0.9 mm, although follicular units may have different approximate diameters as well. The needle may be beveled at the distal end to facilitate easy insertion of the needle into skin. The angle of the bevel may range between 5 degrees and 85 degrees. Other diameters and angles may also be used. The needle may be retracted into the handle or extended from the handle via the needle retractor. For example, a length between the proximal end of the handle and the distal end of the needle may be controlled by the needle retractor. As used herein, chamfered and beveled may be used interchangeably.

In some example embodiments, a tip of the needle may include a bevel on two edges of the tip, or a “double bevel,” and an area above/proximal to the tip that is an “obturating portion.” In some example embodiments, the needle tip has a dual-function of cutting and opening an implantation site. In some example embodiments, a leading edge of the needle is sharp (beveled or ground). Behind the sharp portion of the needle are surfaces for obturating which are polished to be not sharp, filleted, radiused, and otherwise smoothed or deburred for obturating without tissue injury.

The hair transplantation device may include a shuttle that slidably translates inside the lumen of the needle. The shuttle may be cylindrical or any other shape. The shuttle may have an outer diameter between 0.4 mm and 1.5 mm with a lumen diameter between 0.3 mm and 1.4 mm. The shuttle may be beveled at the distal end. When a follicular unit is loaded into the shuttle, the follicular unit may extend into the beveled end to cause the follicular unit to remain in place when the shuttle is retracted. The angle of the bevel may range between 5 degrees and 90 degrees. Other diameters and angles may also be used. The shuttle may be retracted into the needle or extended from the needle via the shuttle retractor. For example, a length between the proximal end of the handle and the distal end of the shuttle may be controlled by the shuttle retractor. The shuttle may be loaded with a follicular unit by the surgeon/operator. For example, the surgeon may manually load a follicular unit into the shuttle using tweezers. The follicular unit may be loaded from the distal end of the shuttle by grasping the follicular unit and sliding the follicular unit into the lumen of the shuttle along a longitudinal slit in the shuttle. At the proximate end of the shuttle, a circumferential slit may allow the tweezers to release from the follicular unit at after the follicular unit is loaded.

The hair transplantation device may include a stabilizer at the proximal end of the shuttle. The stabilizer assists in stabilizing the follicular unit when the shuttle is retracted and the follicular unit is implanted into the implant site. The stabilizer may not push on the follicular unit when the shuttle is retracted but may aid, along with friction between the distal end of the follicular unit and the skin/tissue, in stabilizing the position of the follicular unit as the shuttle is retracted.

The hair transplantation device disclosed herein has many advantages. For example, the disclosed device both prepares implant sites and implants follicular units thereby saving time and increasing the likelihood of successful implantation. The needle opens the skin and creates a protected pathway through the needle for the shuttle to provide a protected delivery of the follicular unit. The protected delivery reduces the risk that the follicular unit will be damaged during implantation. The hair transplantation device controls the depth of the implantation which improves the likelihood that the implantation will be successful. The shuttle protects the follicular unit which improves the likelihood that the implantation will be successful. The follicular unit is not pushed out of the shuttle, but rather friction between the follicular unit extending into the beveled end of the shuttle and the skin along with the stabilizer cause the follicular unit to stay in place. By not pushing on the follicular unit during implantation, the likelihood of damaging the follicular unit during implantation is reduced or eliminated.

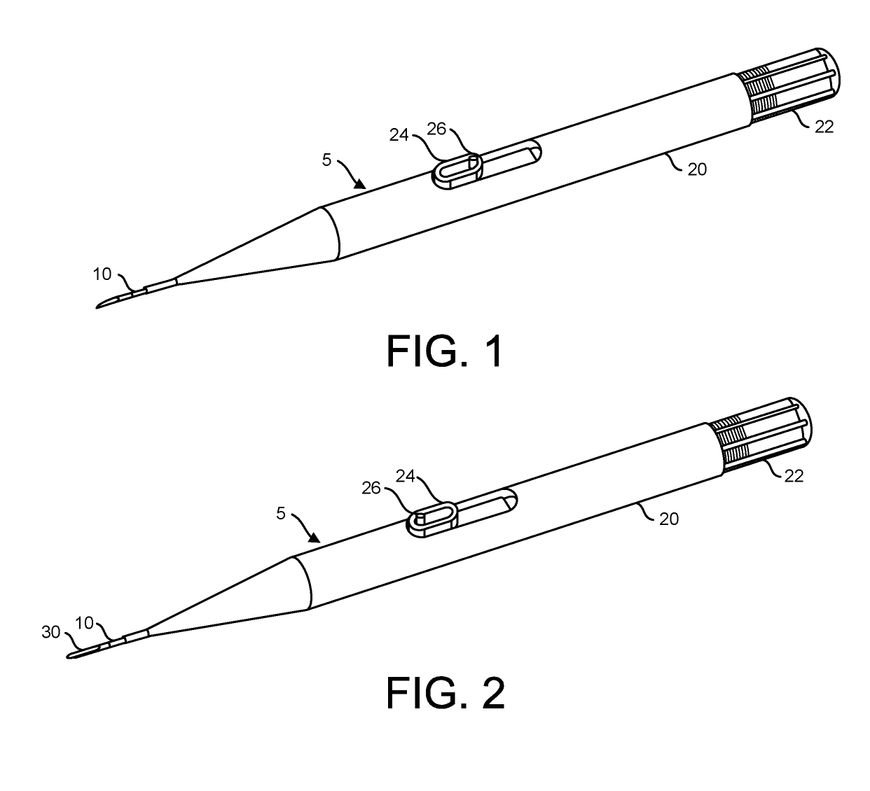

FIG. 1 illustrates an embodiment of a hair transplantation device 5 including a needle 10, a handle 20, a depth adjuster 22, a needle retractor 24, and a shuttle retractor 26. As shown in FIG. 1, the needle 10 can be positioned in an extended position. The hair transplantation device 5 may prepare an implant site with needle 10 at the distal end of the device. A distal end of the needle 10 may be inserted into the scalp or other skin of a patient to a depth determined by a length the needle 10 extends from the distal end of the handle 20. The length at which the needle 10 extends from the handle can be adjusted (e.g., increased, decreased) using a depth adjuster 22 associated with the handle 20. Depth adjuster 22 can be threadably coupled to a body of the handle such that rotation of the depth adjuster 22 relative to the body of the handle causes the needle to retract or extend from the handle. For example, clockwise rotation of the depth adjuster 22 can cause the needle to extend from the handle and counter-clockwise rotation of the depth adjuster 22 can cause the needle to retract into the handle. Although described as being threadably adjustable, the depth adjuster can be adjustable in any number of ways. The inserted needle 10 creates an opening or implant site in the skin and the wall of the needle protects the implant site during implanting of the follicular unit. Needle retractor 24 may include any type of lever or other actuator that causes needle 10 to retract into the handle 20 or to advance from the handle 20.

FIG. 2 illustrates an embodiment of a needle 10 with a shuttle 30 extended to a loading position. The shuttle 30 may include another cannula (in addition to the needle 10) and the shuttle 30 can extend along the inside of the needle 10. Inside shuttle 30 may be a follicular unit for implantation. The shuttle 30 may be beveled at the distal end with the follicular unit extending into the beveled end. The shuttle 30 may be advanced in the needle 10 to deliver the follicular unit to the implant site. The shuttle may then be retracted with shuttle retractor 26.

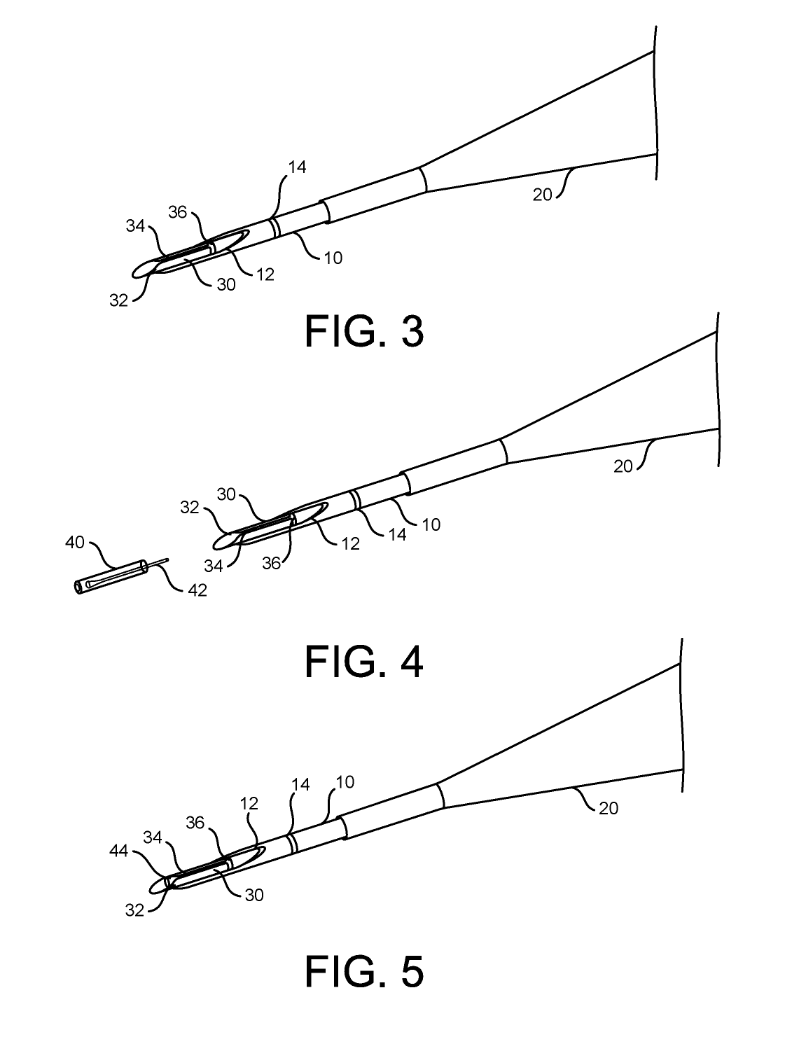

FIG. 3 illustrates a detail view of a shuttle in a loading position. The needle 10 may include beveled end 12 and the beveled end 12 may puncture the skin before implantation of a follicular unit and protect the implant site from closing or being damaged before the follicular unit is implanted. Once a follicular unit is loaded into shuttle 30, the follicular unit may extend into beveled end 32. The shuttle 30 may include a longitudinal slit 34 and a circumferential slit 36. As a follicular unit is being loaded, a follicular unit loading device (e.g., tweezers) may slide along longitudinal slit 34 and release from the follicular unit via circumferential slit 36. In addition, the needle 10 may include one or more depth indicators, such as depth marker 14 that indicates a depth that the needle 10 is inserted into skin.

FIG. 4 illustrates a follicular unit 40, shuttle 30, and needle 10. In FIG. 4, the follicular unit 40 is ready to be loaded into shuttle 30. The follicular unit 40 may be loaded into shuttle 30 with the end where hair 42 exits the follicular unit loaded first into shuttle 30 (e.g., directed at the proximal end of hair transplantation device 5). The end of the follicular unit 40 opposite the hair may align with the beveled end of the shuttle 30. In some example embodiments, friction between follicular unit 40 and the interior wall of shuttle 30 will hold the follicular unit in the device 5 until time for delivery.

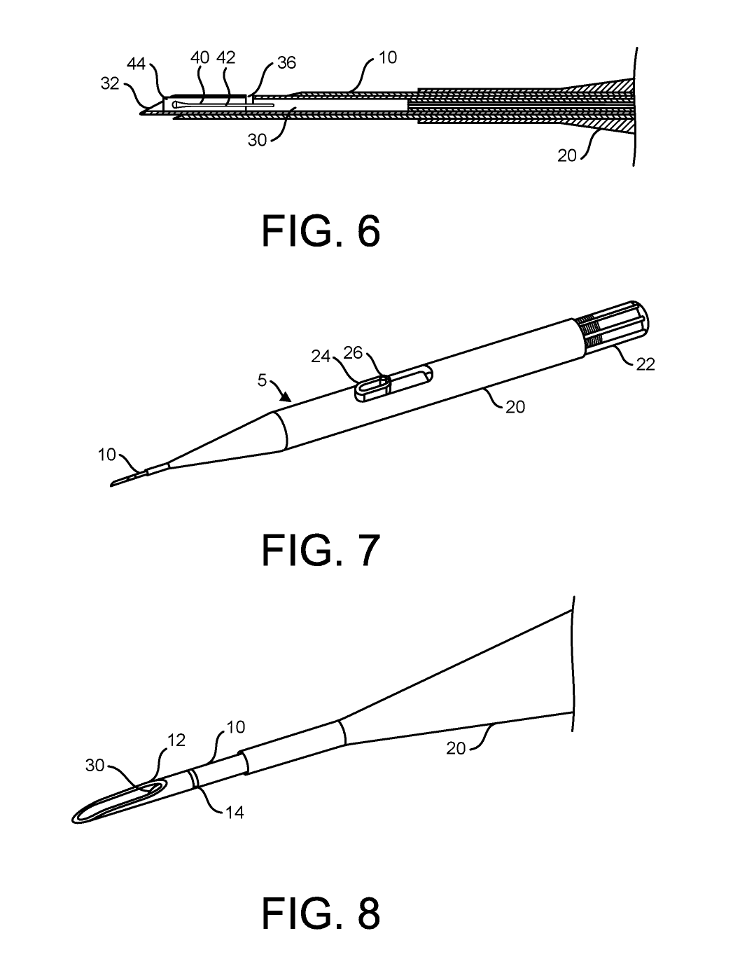

FIGS. 5 and 6 illustrate a follicular unit 40 loaded into shuttle 30 and needle 10. As described above, the follicular unit 40 is positioned such that the end where hair 42 extends from the follicular unit 40 is loaded farthest into shuttle 30. The end of follicular unit 40 opposite to the hair may extend at 44 into bevel 32. Friction between the follicular unit extending into bevel 32 of the shuttle 30 and the implant site may hold the follicular unit 40 in place while the shuttle 30 is retracted.

FIGS. 7-9 illustrate the hair transplantation device 5 configured such that the shuttle is retracted into the needle 10. The shuttle 30 may be retracted so that the distal end of the shuttle is positioned proximal to the bevel 12 of needle 10. In some embodiments, the needle 10 may have a two-step bevel 12 and 12A. In some embodiments, the needle can include a single continuous bevel 12.

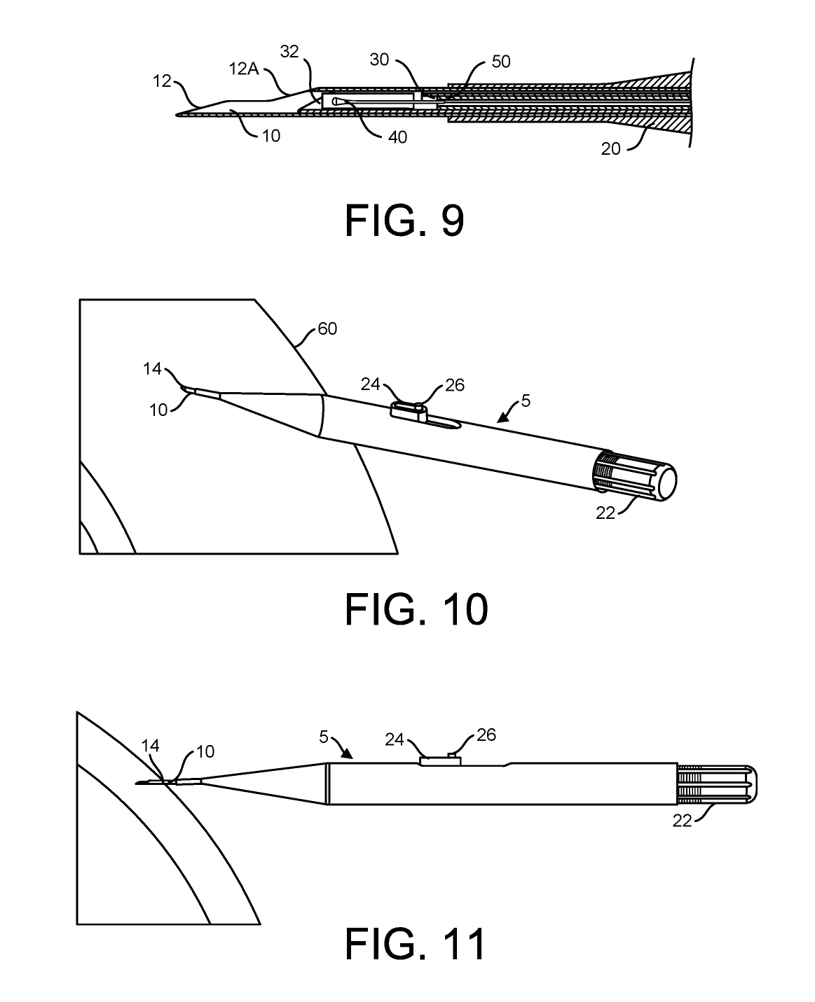

As shown in FIG. 9, the follicular unit 40 can be loaded into shuttle 30 such that a distal end of the follicular unit 40 extends along a part of the bevel 32 of shuttle 30. The stabilizer 50, which can extend along a lumen of the shuttle 30, may be positioned proximal to the follicular unit and relative to the beveled end of the shuttle. Stabilizer 50 may stabilize the follicular unit 40 as the shuttle 30 is retracted and the follicular unit 40 is implanted in skin. Stabilizer 50 may aide implantation of the follicular unit 40 into the implantation site.

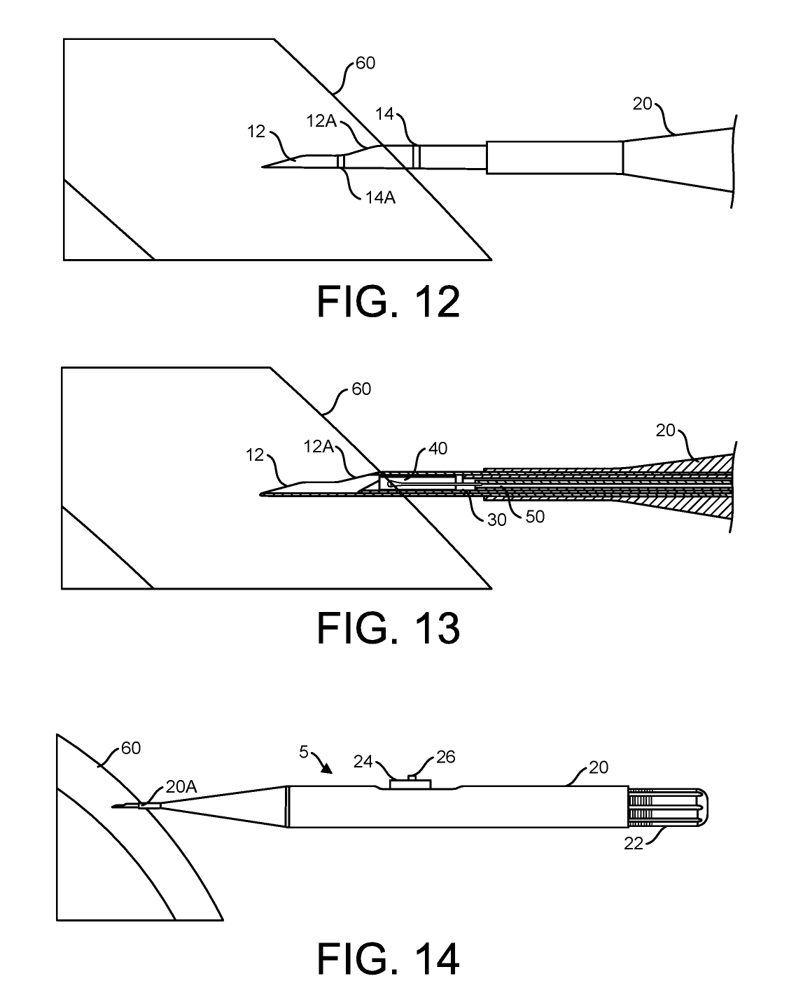

FIGS. 10-13 illustrate the distal end of a needle 10 inserted into skin 60. The needle 10 may be inserted into the skin 60 to a depth indicated by a depth marker 14. In addition, the needle 10 may create and maintain an opening (e.g., implantation site) in the skin 60 for a follicular unit to be implanted.

In some embodiments, the angle of the bevel 12 along the needle 10 may be selected to cause cutting of tissue and easy insertion of the needle 10 into the skin. In some embodiments, a second bevel 12A angle may be selected to facilitate obturating the tissue. In some embodiments, multiple depth markers may be included on an outer surface of the needle 10 to assist the user in determining a depth at which to insert the needle 10 into the skin. For example, a first marker 14A, when aligned with a surface of the skin, can indicate to the user that the needle 10 is inserted 3 mm into skin 60. Similarly, a second marker 14 can be used to indicate to the user that the needle is inserted 6 mm into skin 60.

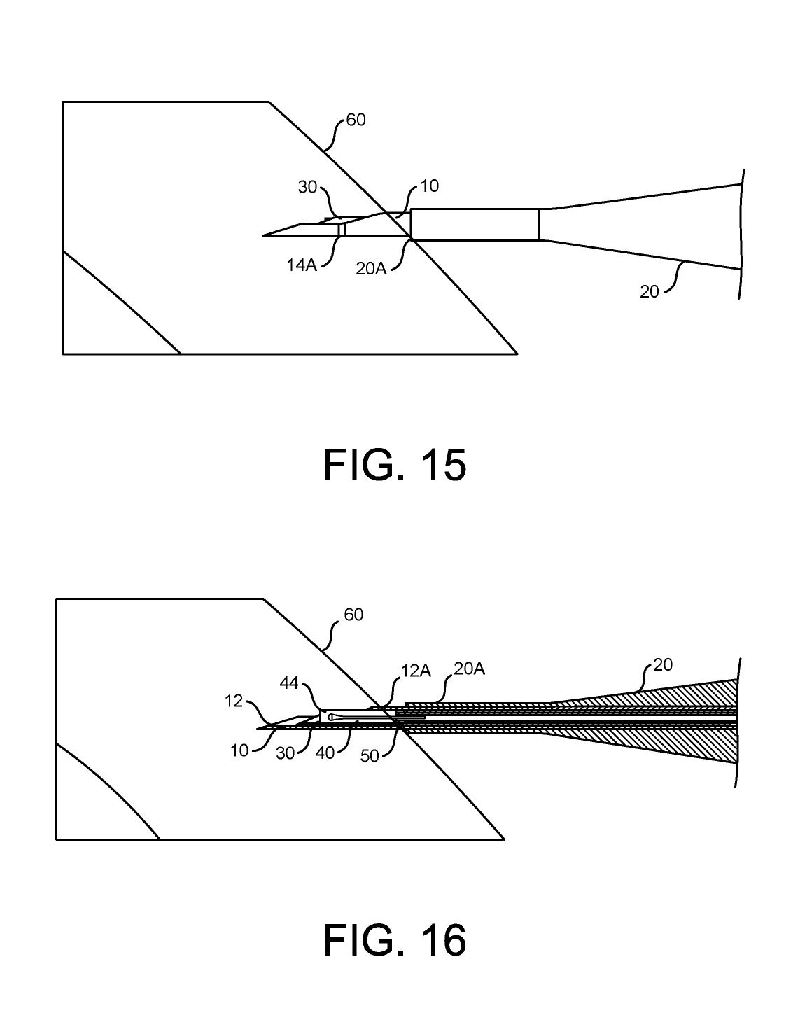

FIGS. 14-16 illustrate a follicular unit 40 advanced into the skin 60 with a distal end of the handle 20 at 20A is positioned adjacent to the surface of the skin 60. As shown in FIG. 14, the shuttle retractor 26 may be actuated to advance the shuttle into the opening in the skin (e.g., implantation site) made by needle 10.

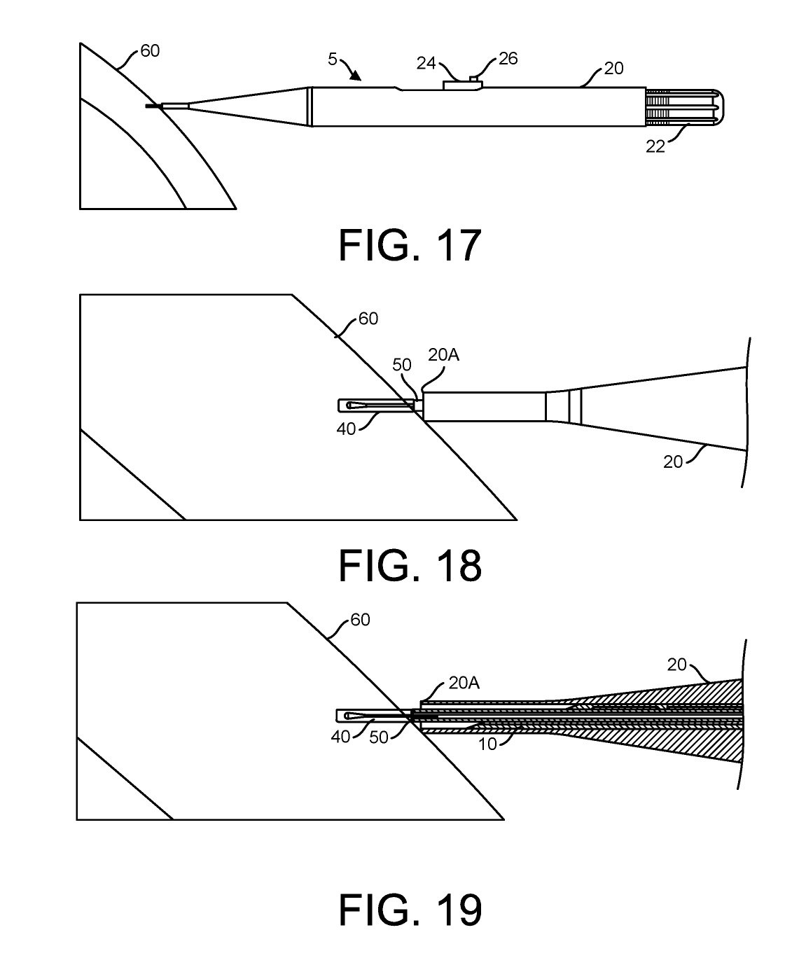

FIGS. 17-19 illustrate the follicular unit 40 implanted into the implant site. FIGS. 17-19 show the shuttle 30 retracted into needle 10, and needle 10 (with shuttle 30) retracted into the handle 20. The shuttle retractor 26 may be retracted by actuating retractor 26 in the opposite direction from the direction that advanced the shuttle 30 into skin 60. Needle retractor 24 may be retracted into the needle 10 and shuttle 30 by actuating retractor 24 in the opposite direction from the direction that advanced the needle 10 into skin 60. As shown in FIG. 18, the stabilizer 50 and outer sheath 20A may be fixed in position while the needle 10 and shuttle 30 are retracted.

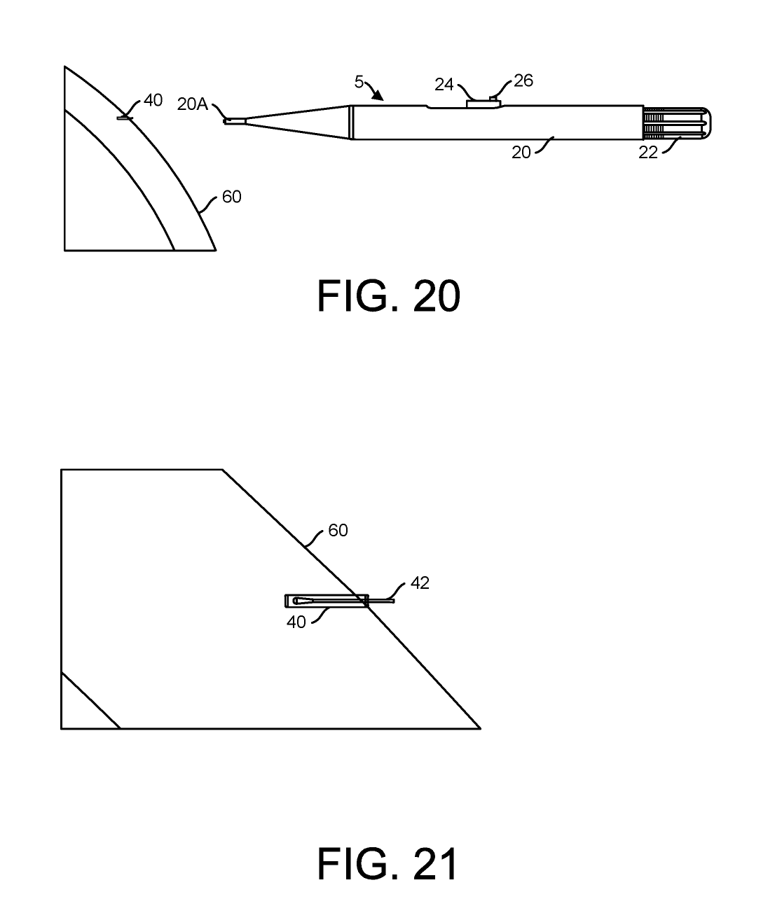

FIGS. 20-21 illustrate the follicular unit implanted into the implantation site in the skin 60. In addition, as shown in FIG. 20, after implantation of the follicular unit 40, the needle 10 can be retracted into the housing of the hair implantation device 5 and the hair implantation device 5 can be removed from contact with the patient's skin 60.

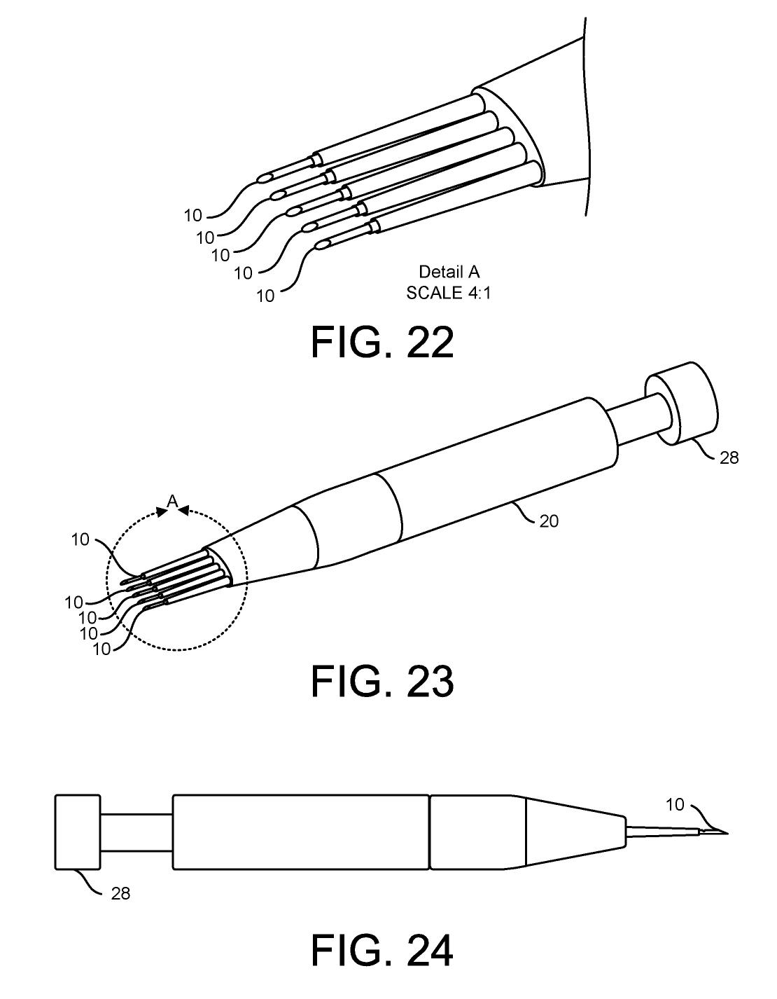

FIGS. 22-24 illustrate another embodiment of the hair transplantation device 5 with multiple needles 10 extending from the body of the housing 20. In some embodiments, multiple implant sites may be prepared and multiple follicular units may be implanted per use of the device. In some example embodiments, the multiple needles may be arranged in handle in a linear array as shown in FIGS. 22-23. In other embodiments, needles 10 may be arranged in a matrix with a predetermined quantity of rows and a predetermined quantity of columns. For example, a hair transplantation device may include five rows and five columns, thus including twenty-five needles. Each needle 10 may operate in accordance with the foregoing description of a single needle device in FIGS. 1-21. When the hair transplantation device includes multiple needles, the device may prepare the entire array or matrix of implant sites followed by the entire array or matrix implanting a plurality of follicular unit (e.g., one follicular unit implanted in each of the implant sites created by the matrix of needles). For example, a device with a five-by-five matrix may prepare twenty-five implant sites followed by implanting twenty-five follicular units. In some example embodiments, a spring-loaded actuator 28 may cause implantation of the array or matrix of follicular units.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The implementations set forth in the foregoing description do not represent all implementations consistent with the subject matter described herein. Instead, they are merely some examples consistent with aspects related to the described subject matter. Although a few variations have been described in detail herein, other modifications or additions are possible. In particular, further features and/or variations can be provided in addition to those set forth herein. For example, the implementations described above can be directed to various combinations and sub-combinations of the disclosed features and/or combinations and sub-combinations of one or more features further to those disclosed herein. In addition, the logic flows depicted in the accompanying figures and/or described herein do not necessarily require the particular order shown, or sequential order, to achieve desirable results. The scope of the following claims may include other implementations or embodiments.

What is claimed:

1. A device for transplanting hair comprising:
a handle;
a needle extending from the handle and including a beveled distal end for creating an implant site in skin, wherein the implant site is configured to receive a follicular unit having at least one hair follicle;
a shuttle tube that extends from the handle along a first lumen of the needle, wherein the shuttle tube is configured to capture the follicular unit for implanting in the implant site; and
a stabilizer that extends from the handle along a second lumen of the shuttle tube, wherein the stabilizer stabilizes the follicular unit in the implant site.

2. The apparatus of claim 1, wherein the needle includes markings along a circumference of the needle to indicate a needle insertion depth in the skin.

3. The apparatus of claim 1, wherein the beveled distal end includes a first beveled angle extending a first distance from the distal end and a second beveled angle extending a second distance from a proximal end of the first beveled angle.

4. The apparatus of claim 1, wherein a distal end of the handle includes a dial to threadably control a length the needle extends from the handle.

5. The apparatus of claim 1, wherein the shuttle tube includes a longitudinal opening extending a length from a distal end of the shuttle tube, and wherein the longitudinal opening is shaped to allow the follicular unit being grasped by a grasping device to be loaded within the second lumen of the shuttle tube through the longitudinal opening.

6. The apparatus of claim 5, wherein the shuttle tube includes a transverse opening extending a partial circumference around the shuttle tube for allowing release of the grasping device from the follicular unit after loading the follicular unit in the second lumen of the shuttle tube.

7. The apparatus of claim 1, further comprising more than one needle to implant more than one follicular unit at approximately the same time.

8. The apparatus of claim 1, wherein the needle retracts the skin to protect the implant site during the implanting of the follicular unit.

9. The apparatus of claim 1, wherein the apparatus is attached to a medical robot or a hair transplantation robot.
